(19) United States
(12) Patent Application Publication
(10) Pub. No.: US 2007/0218135 A1
(43) Pub. Date: Sep. 20, 2007

(54) Sustained release matrix pharmaceutical composition

(75) Inventors: Amit Mukharya, Colaba (IN); Abhaykumar M. Chandak, Airoli (IN); Anandi Krishnan, Vashi (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/717,980

(22) Filed: Mar. 14, 2007

Related U.S. Application Data
(60) Provisional application No. 60/832,379, filed on Jul. 21, 2006.

(30) Foreign Application Priority Data
Mar. 14, 2006 (IN) 366/MUM/2006

(51) Int. Cl. A61K 9/24

(52) U.S. Cl. 424/471

(57) ABSTRACT

A sustained release pharmaceutical composition in solid dosage form is provided comprising (a) a therapeutically effective amount of one or more active pharmaceutical agents; (b) a first high viscosity release retarding cellulose ether; and (c) a second high viscosity release retarding cellulose ether, wherein the first and second high viscosity release retarding cellulose ethers are of the same material.

### PRIORITY

This application claims the benefit under 35 U.S.C. §119 to U.S. Provisional Application No. 60/832,379, filed on Jul. 21, 2006, and entitled “PHARMACEUTICAL SUSTAINED RELEASE MATRIX COMPOSITION” and to Indian Provisional Application No. 366/MUM/2006, filed on Mar. 14, 2006, and entitled “PHARMACEUTICAL SUSTAINED RELEASE MATRIX COMPOSITION”, the contents of each of which are incorporated by reference herein.

### BACKGROUND OF THE INVENTION

1. Technical Field

The present invention generally relates to a sustained release pharmaceutical compositions containing an active pharmaceutical ingredient such as alprazolam.

2. Description of Related Art

Alprazolam, also known as 8-chloro-1-methyl-6-phenyl-4H-s-triazolo[4,3-α][1,4]benzodiazepine, is a member of the 1,4-benzodiazepine class and can be represented by the structure of Formula I: 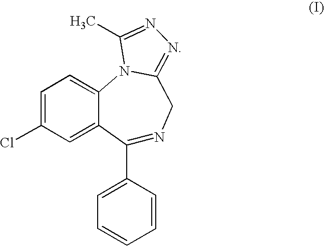
Alprazolam is marketed in an extended release tablet form under the tradename Xanax XR®. Xanax XR® is indicated for the treatment of panic disorder with or without agoraphobia. See, e.g., The Merck Index, Thirteenth Edition, 2001, p. 310-11, monograph 310; and Physician's Desk Reference, “Xanax XR”, 60th Edition, pp. 2655-2659 (2005).

Acetazolamide, also known as N-(5-sulfamoyl-1,3,4-thiadiazol-2-yl)acetamide, can be represented by the structure of Formula II: 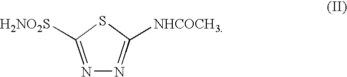
Acetazolamide is an inhibitor of the enzyme carbonic anhydrase. Acetazolamide is indicated for the adjunctive treatment of: edema due to congestive heart failure; drug-induced edema; centrencephalic epilepsies (petit mal, unlocalized seizures); chronic simple (open-angle) glaucoma, secondary glaucoma and preoperatively in acute angle-closure glaucoma where delay of surgery is desired to lower intraocular pressure. Acetazolamide is marketed under the trade name Diamox®. See, e.g., The Merck Index, Thirteenth Edition, 2001, p. 11, monograph 54.

Sustained-release alprazolam formulations have been investigated, including formulations wherein alprazolam is dispersed in a polymer matrix, for example, a hydroxypropylmethylcellulose (HPMC) matrix. Franz et al. (1987), Journal of Controlled Release 5, 159-172, examined effects of several formulation variables on in vitro alprazolam release rate from such a matrix formulation comprising HPMCs of different viscosity grades, sodium carboxymethylcellulose (sodium CMC) and lactose. These variables included ratio of high to low viscosity HPMC, ratio of sodium CMC to lactose, and matrix drug loading. Franz et al. established relationships among these variables, but failed to provide guidance on absolute amounts of HPMC to be formulated with desired amounts of alprazolam.

U.S. Patent Application Publication No. 20040006072 (“the '072 application”) discloses a sustained release pharmaceutical composition in a form of a tablet of alprazolam containing a high viscosity HPMC and a low viscosity HPMC, wherein the total weight of HPMC in the composition varies from 110 mg to 135 mg per tablet. The '072 application further discloses that the high and low viscosity HPMCs were used in a weight ratio of about 40:60 to about 60:40. The high viscosity HPMC used in the '072 application has a viscosity of about 3000 to about 5600 cP whereas the low viscosity HPMC has a viscosity of about 2 to about 400 cP, when measured in a 2% aqueous solution at 20° C.

The prior art reveals that considerable efforts have been made to develop a sustained release composition of alprazolam, using a blend of low viscosity HPMC and high viscosity HPMC. However, there remains a need for improved sustained release compositions of active pharmaceutical ingredients such as alprazolam.

### SUMMARY OF THE INVENTION

In accordance with one embodiment of the present invention, a sustained release pharmaceutical composition in solid dosage form is provided comprising (a) a therapeutically effective amount of one or more active pharmaceutical ingredients; (b) a first high viscosity release retarding cellulose ether; and (c) a second high viscosity release retarding cellulose ether, wherein the first and second high viscosity release retarding cellulose ethers are of the same material.

In accordance with a second embodiment of the present invention, a sustained release pharmaceutical composition in solid dosage form is provided comprising a therapeutically effective amount of one or more active pharmaceutical ingredients dispersed in a matrix comprising (a) a first high viscosity release retarding cellulose ether; and (b) a second high viscosity release retarding cellulose ether, wherein the first and second high viscosity release retarding cellulose ethers are of the same material and the viscosity of the first high viscosity release retarding cellulose ether is different than the viscosity of the second high viscosity release retarding cellulose ether.

In accordance with a third embodiment of the present invention, a sustained release pharmaceutical composition in solid dosage form is provided comprising (a) a therapeutically effective amount of one or more active pharmaceutical ingredients; (b) a first high viscosity hydroxypropyl methylcellulose (HPMC); and (c) a second high viscosity HPMC.

In accordance with a fourth embodiment of the present invention, a method of treating a CNS condition or disorder in a subject is provided, the method comprising orally administering to the subject a therapeutically effective amount of a sustained release pharmaceutical composition in solid dosage form is provided comprising (a) a therapeutically effective amount of one or more active pharmaceutical ingredients; (b) a first high viscosity release retarding cellulose ether; and (c) a second high viscosity release retarding cellulose ether, wherein the first and second high viscosity release retarding cellulose ethers are of the same material.

### DETAILED DESCRIPTION OF THE PREFERED EMBODIMENTS

The present invention is directed to a sustained release pharmaceutical composition in solid dosage form which includes a therapeutically effective amount of one or more active pharmaceutical ingredients and at least two high viscosity high viscosity release retarding cellulose ethers of the same release retarding material.

Suitable active pharmaceutical ingredients for use in the compositions of the present invention include, but are not limited to, alprazolam, acetazolamide and the like and mixtures thereof. Illustrative methods for preparation of alprazolam, 8-chloro-1-methyl-6-phenyl-4H-s-triazolo-[4,3-.alpha.]-1,4-benzodiazepine (I), are known and disclosed in, for example, U.S. Pat. Nos. 3,709,898; 3,879,413; 3,980,789; and 3,987,052, the contents of which are incorporated by reference herein. Any pharmaceutically acceptable form of alprazolam or acetazolamide can be used, including any suitable crystalline or other solid state form, enantiomer or tautomer thereof.

In the pharmaceutical composition in solid dosage forms of the present invention such as a tablet, the active pharmaceutical ingredient such as alprazolam can be present in an amount of about 0.1 mg to about 5 mg, preferably about 0.5 to about 3 mg, for example about 0.5 mg, about 1 mg, about 2 mg or about 3 mg. In one embodiment, the weight for all the quantities of the solid dosage form, i.e., a total solid dosage form (e.g., tablet) weight, can range from about 200 mg to about 500 mg, preferably from about 300 mg to about 400 mg. with 300 mg being most preferred.

The active pharmaceutical ingredient(s) is distributed in a matrix that comprises at least a first high viscosity release retarding cellulose ether and a second high viscosity release retarding cellulose ether, wherein the first and second high viscosity release retarding cellulose ethers are of the same material. In one embodiment, the viscosity of the first high viscosity release retarding cellulose ether is different than the viscosity of the second high viscosity release retarding cellulose ether. In another embodiment, the viscosity of the first high viscosity release retarding cellulose ether is the same as the viscosity of the second high viscosity release retarding cellulose ether and wherein the substitution type of each of the cellulose ether is different. The term “high viscosity release retarding cellulose ethers” as used herein are those cellulose ethers which tend to slow down or retard or delays the release of the active ingredient after administration and have a viscosity greater than about 400 centipoise (cP) and preferably at least about 1500 cP and most preferably at least about 4000 cP as measured in a 2% aqueous solution at 20° C. In one embodiment, the first high viscosity release retarding cellulose ether can have a viscosity greater than or equal to about 1000 cP and the second high viscosity release retarding cellulose ether can have a viscosity greater than or equal to about 5000 cP. In a second embodiment, the first high viscosity release retarding cellulose ether can have a viscosity greater than or equal to about 3500 cP and the second high viscosity release retarding cellulose ether can have a viscosity greater than or equal to about 10,000 cP. In a third embodiment, the first high viscosity release retarding cellulose ether can have a viscosity greater than or equal to about 4000 cP and the second high viscosity release retarding cellulose ether can have a viscosity greater than or equal to about 15,000 cP.

Suitable release retarding cellulose ethers for use in the compositions of the present invention include, but are not limited to, hydroxypropyl methylcellulose (HPMC) polymers, hydroxypropyl cellulose (HPC) polymers, hydroxyethyl cellulose (HEC) polymers, and the like. Preferably, the first and second release retarding cellulose ethers are a HPMC polymers. They are all commercially available in a wide variety of viscosity grades.

HPMC polymers are commercially available in different viscosity grades, under several trade names, including Methocel® E, F, J and K of Dow Chemical Co., U.S.A., HPM of British Celanese Ltd., U.K., and Metalose® of Shin-Etsu Ltd., Japan. The various grades available under a given trade name typically represent differences in methoxy and hydroxypropoxy content as well as molecular weight of the HPMC. The cellulose ethers routinely used in pharmaceutical industry for sustained release matrix compositions, differ with respect to the solubility, viscosity, gelling strength and hydration rate. The selection of a desired grade of a cellulose ether is based on the aforementioned properties of the cellulose ether and characteristics of the active ingredient. The viscosity of HPMC polymers affects hydration speed in the initial stage of the exposure to the dissolution medium and the gel strength or erosion rate of the gel in the subsequent stages of exposure.

The high viscosity grades of HPMC polymers in the composition of the present invention are available in various substitution types. The substitution type of HPMC polymers affects hydration speed of HPMC particles and gel strength, which can influence the dissolution profile. The term “high viscosity HPMC” herein refers to HPMC having a viscosity of about 1,500 to about 225,000 cP. Representative high viscosity HPMCs of various substitution types for use herein include HPMC 2208, HPMC 2910, and HPMC 2906. A high viscosity HPMC 2208 can have a viscosity of about 3000 to about 5600 cP (e.g., available as Methocel® K4 MP of Dow) or a viscosity of about 11,250 to about 21,000 cP (e.g., available as Methocel® K15M of Dow) and contains about 19% to about 24% by weight of methoxyl substituents, and about 7% to about 12% by weight of hydroxypropoxyl substituents. HPMC 2910 can have a viscosity of about 3000 to about 5600 cP (e.g., available as Methocel® E4M of Dow) and contains about 28% to about 30% by weight of methoxyl substituents, and about 7% to about 12% by weight of hydroxypropoxyl substituents. HPMC 2906 can have a viscosity of about 3000 to about 5600 cP (e.g., available as Methocel® F4M of Dow) and contains about 27% to about 30% by weight of methoxyl substituents, and about 4% to about 7.5% by weight of hydroxypropoxyl substituents.

In one embodiment of the present invention, the two different high viscosity HPMC polymers are about 4000 cP and about 15000 cP wherein the HPMC is the same or different substitution type. In a preferred embodiment of the present invention, the two different high viscosities HPMCs are 4000 cP and 15000 cP wherein the substitution types are 2208 and 2910, respectively. In another embodiment, the two different high viscosity HPMC polymers can have the same viscosity and be of a different substitution type as exemplified in Example 3.

In one embodiment, a sustained release matrix composition of the present invention comprises two different high viscosity grades HPMC polymers, wherein the viscosity of both the grades, is not less than about 400 cp, preferably not less than about 1500 cp (centipoise), more preferably not less than about 4000 cp. The two different high viscosity grades of HPMC may belong to the same or different substitution types.

Generally, the first high viscosity release retarding cellulose ether can be present in the solid dosage forms of the present invention in an amount ranging from about 15 to about 35 weight percent and preferably from about 20 to about 25 weight percent while the second high viscosity release retarding cellulose ether can be present in the solid dosage forms of the present invention in an amount ranging from about 5 to about 20 weight percent and preferably from about 5 to about 15 weight percent.

The solid dosage forms of the present invention are orally applicable single unit dosage forms. Examples of such dosage forms include pills, capsules and tablets. Because of their ease in administration, tablets represent the most advantageous oral solid dosage form. Preferably, the tablet comprises one or more additional pharmaceutically acceptable excipients other than the release retarding cellulose ethers. Such excipients include conventional pharmaceutical tablet excipients such as, for example filler, binders, glidants, lubricants, pH modifying agents, coloring agents, antioxidants, disintegrants, fillers and the like and mixtures thereof. Suitable fillers include, but are not limited to, sugar and sugar alcohols, and the like and mixtures thereof. A preferred sugar is lactose and a preferred sugar alcohol is mannitol. The lactose used in the compositions herein may be lactose monohydrate or anhydrous lactose.

Suitable glidants include colloidal silicon dioxide, and the like and mixtures thereof. Suitable lubricants include magnesium stearate and the like and mixtures thereof.

Optionally the tablets of the present invention may be colored with one or more coloring agents. Selection of coloring agents can be made, for example, so that tablets of different dosage strengths can be easily distinguished. Illustratively, D&C Yellow #10 can be present in an amount of about 0.2 mg to about 0.3 mg per tablet, and/or FD&C Blue #2 can be present in an amount of about 0.05 mg to about 0.09 mg per tablet. In one embodiment, D&C Yellow #10 and FD&C Blue #2 are used in combination as a coloring agent.

The compositions of the present invention provide a release rate of the one or more active pharmaceutical ingredients such as alprazolam that is acceptable for once or twice daily dosing in humans. Thus, the compositions of the present invention can be orally administered in a therapeutically effective amount to a human subject one or two times per day. The compositions of the present invention can be used to treat any condition or disorder that is responsive to benzodiazepine drugs, and are especially useful in treatment or management of general anxiety disorder, anxiety associated with depression, panic disorder and panic attacks.

Another aspect of the present invention provides a method of treating a CNS condition or disorder in a subject. The method comprises orally administering to the subject a therapeutically effective amount of a sustained-release pharmaceutical composition as provided herein. The composition provides a release rate of the active pharmaceutical ingredient such as alprazolam that is acceptable for once or twice daily dosing in humans, thus in a preferred method a composition of the invention is orally administered in a therapeutically effective amount to a human subject one or two times per day.

CNS conditions and disorders include those having a neurologic and/or a psychiatric component. Illustrative CNS conditions and disorders include, for example, personality disorders including paranoid, schizoid, schizotypal, bipolar, histrionic, delusional, narcissistic, emotionally unstable, psychopathic and sociopathic personality disorders; habit and impulse disorders including pathological gambling, stealing, trichotillomania, etc.; obsessive-compulsive disorder; passive-aggressive disorder; acute and transient psychotic disorders; psychotic depression; schizoaffective disorder; hypochondria; cyclothymia; dysthymia; manic-depressive illness; major depressive disorder; treatment-resistant depression; adult and childhood onset schizophrenias; drug dependence including harmful use and abuse of, addiction to or dependence on opioids, narcotics, barbiturates, alcohol, benzodiazepines, amphetamines, cocaine, cannabinoids, hallucinogens, stimulants, nicotine (tobacco) and solvents; withdrawal states and mood and psychotic disorders related to such dependence; sexual dysfunction including hypoactive sexual desire disorder, sexual aversion or avoidance and erectile dysfunction; gender identity disorders; sexual preference disorders; general anxiety disorder; social anxiety disorder; mixed anxiety and depressive disorder; attention deficit hyperactivity disorder (ADHD) and depression and anxiety associated therewith; depression, anxiety, emotional dysregulation and behavioral disturbances associated with mental retardation; developmental disorders including autism, Asperger's syndrome and Rett's syndrome; childhood conduct and attachment disorders; premenstrual dysphoric disorder; postpartum depression; phobias including social phobias, agoraphobia and specific phobias related for example to hospitals, injections, venesection, etc.; posttraumatic stress disorder; dissociative disorder; Briquet's syndrome; affective disorders including depression, bipolar affective disorder and recurrent depressive disorder; organic mood, anxiety and emotionally labile disorders resulting for example from brain damage or dysfunction arising from head injury, intracranial masses, stroke, etc.; chronic fatigue; stress-induced psychotic episodes; dementia including presenile dementia, Pick's disease, vascular dementia, multi-infarct dementia, Alzheimer's disease, dementia associated with Creutzfeldt-Jakob disease and HIV-related dementia; other neurodegenerative disorders including Parkinson's disease and Huntington's disease; suicidal behavior; eating disorders including anorexia, bulimia and binge eating disorder; adjustment disorders; somatization disorder; somatoform autonomic dysfunction; somatoform pain disorder; panic attacks; panic disorder; amnesia; neuropathic pain; fibromyalgia; migraine; epilepsy; tinnitus; enuresis; sleep disorders including insomnia, hypersomnia, narcolepsy, nightmares and night terrors; delirium; postconcussion syndrome; multiple sclerosis; tremors; muscular spasms; restless legs syndrome; Lennox-Gastaut syndrome; motor and vocal tic disorders; Tourette's syndrome; supranuclear palsy; Shy-Drager syndrome; trigeminal neuralgia; Bell's palsy; motor neuron diseases such as amyotrophic lateral sclerosis; and psychosomatic and psychosocial conditions associated with non-CNS disorders such as diabetes, inflammatory disease, infertility, allergies, psoriasis, asthma, hypertension, overactive bladder, thyroid disorders, obesity, immune disorders and cancer.

The following examples are provided to enable one skilled in the art to practice ntion and are merely illustrative of the invention. The examples should not be read as the scope of the invention as defined in the claims.

### EXAMPLE 1

Tablets having the compositions shown in Table 1 were prepared with an aoprazolam content of 0.5 mg, 1 mg, 2 mg and 3 mg, respectively.

TABLE 1

| | | 0.5 mg | 1 mg | 2 mg | 3 mg |
|---|---|---|---|---|---|
| Sr. | | Qty/Tab | Qty/Tab | Qty/Tab | Qty/Tab |
| No. | Ingredient | (mg) | (mg) | (mg) | (mg) |
| 1 | Alprazolam | 0.50 | 1.00 | 2.00 | 3.00 |
| 2 | Lactose monohydrate | 147.50 | 146.80 | 145.80 | 144.60 |
| | (Pharmatose ® 200M) | | | | |
| 3 | Hydroxypropyl | 60.00 | 60.00 | 60.00 | 60.00 |
| | methylcellulose | | | | |
| | Substitution type 2910 | | | | |
| | (Methocel ® E4M) | | | | |
| | Viscosity by Ubbelhode | | | | |
| | 3000-5600 cps | | | | |
| | (nominal value 4000 cps) | | | | |
| 4 | Hydroxypropyl | 28.00 | 28.00 | 28.00 | 28.00 |
| | methylcellulose | | | | |
| | Substitution type 2208 | | | | |
| | (Methocel ® K15M) | | | | |
| | Viscosity by Ubbelhode | | | | |
| | 11,250-21,000 cps | | | | |
| | (nominal value 15,000 cps) | | | | |
| 5 | Povidone K 25 | 8.00 | 8.00 | 8.00 | 8.00 |
| 6 | D&C yellow No. 10 | — | 0.20 | — | 0.20 |
| 7 | FD&C blue No. 2 | — | — | 0.20 | 0.20 |
| | Isopropyl alcohol (IPA) | q.s | q.s | q.s | q.s |
| 8 | Lactose Monohydrate | 50.00 | 50.00 | 50.00 | 50.00 |
| | (Pharmatose ® DCL 11) | | | | |
| 9 | Colloidal silicon dioxide | 3.00 | 3.00 | 3.00 | 3.00 |
| 10 | Magnesium stearate | 3.00 | 3.00 | 3.00 | 3.00 |
| | Total | 300.00 | 300.00 | 300.00 | 300.00 |

Procedure

The alprazolam and ingredient numbers 2, 3, 4, 6 and 7 were passed through an American Society for Testing and Materials (ASTM) mesh 60# and mixed uniformly. Next, povidone was dissolved in isopropyl alcohol (IPA) and the dry blend was granulated with IPA. The wet mass was air dried in a fluid bed drier for 15 minutes and then further dried at 55° C. until the loss on drying (LOD) was found to be less than 3%. The dried granules were sifted through ASTM mesh # 30. The sifted granules were blended for 10 minutes with lactose monohydrate (Pharmatose DCL 11) and colloidal silicon dioxide which were previously sifted through ASTM mesh #40. The final lubrication was done for 5 minutes with magnesium stearate, which was previously sifted through ASTM mesh # 80. The lubricated granules were then compressed into tablets.

Testing

A study was conducted of the drug release of the 3 mg alprazolam tablets of Example 1 compared to 3 mg alprazolam Xanax® XR tablets (Pharmacia Corporation) in a dissolution bath at 37±0.5° C. in 500 ml of a pH 6.0 Phosphate Buffer using a U.S. Pharmacopoeia (USP) Type I apparatus with a basket speed of 100 rpm for 18 hours. The results are set forth below in Table 2.

TABLE 2

| | | |
|---|---|---|
| Time | Example 1 | Xanax ® XR |

| (Hours) | (3 mg) | Lot # 1 (3 mg) | Lot # 2 (3 mg) |
|---|---|---|---|
| 0.5 | 2 | 9 | 7 |
| 1 | 19 | 14 | 11 |
| 2 | 29 | 22 | 19 |
| 4 | 39 | 37 | 32 |
| 6 | 47 | 49 | 44 |
| 8 | 57 | 58 | 54 |
| 10 | 62 | 67 | 63 |
| 12 | 67 | 74 | 71 |
| 14 | 71 | 79 | 77 |
| 16 | 75 | 85 | 82 |

A study was also conducted of the drug release of the 3 mg alprazolam tablets of Example 1 compared to 3 mg alprazolam Xanax® XR tablets (Pharmacia Corporation) in a dissolution bath at 37±0.5° C. in 500 ml of a pH 6.0 Phosphate Buffer using a U.S. Pharmacopoeia (USP) Type I apparatus with a basket speed of 100 rpm for two hours, then a dissolution bath at 37±0.5° C. in 500 ml of a 4.5 pH acetate buffer using a USP Type I apparatus with a basket speed of 100 rpm for the next two hours, and followed by dissolution bath at 37±0.5° C. in 500 ml of a 6.8 pH phosphate buffer using a USP Type I apparatus with a paddle speed of 100 rpm for the next 16 hours. The results are set forth below in Table 3.

TABLE 3

Xanax ® XR

| | Time | Example 1 | Lot #1 | Lot #1 |
|---|---|---|---|---|
| | (Hours) | (3 mg) | (3 mg) | (3 mg) |
| 0.1N HCl | 0.5 | 19 | 17 | 14 |
| | 1 | 32 | 26 | 24 |
| | 2 | 47 | 40 | 39 |
| pH 4.5 Acetate | 3 | 56 | 52 | 51 |
| Buffer | 4 | 59 | 54 | 53 |
| pH 6.8 | 5 | 63 | 58 | 57 |
| Phosphate | 6 | 65 | 62 | 60 |
| Buffer | 8 | 69 | 71 | 69 |
| | 10 | 73 | 80 | 77 |
| | 12 | 76 | 87 | 84 |
| | 14 | 80 | 92 | 89 |
| | 16 | 83 | 95 | 93 |

### EXAMPLE 2

Tablets having the composition shown in Table 4 were prepared in substantially the same as in Example 1.

TABLE 4

| | Qty/Tab |
|---|---|
| INGREDIENTS | (mg) |
| Alprazolam | 3.00 |
| Pharmatose 200M | 137.60 |
| Hydroxypropyl methylcellulose | 65.00 |
| (Methocel ® K4M CR) | |
| Viscosity by Ubbelhode | |
| 3000-5600 cps | |
| (nominal value 4000 cps) | |
| Hydroxypropyl methylcellulose | 35.00 |
| (Methocel ® K15M) | |
| Viscosity by Ubbelhode | |
| 11,250-21,000 cps | |
| (nominal value 15,000 cps) | |
| D&C Yellow No. 10 | 0.20 |
| FD&C Blue No. 2 | 0.20 |
| Povidone K-25 | 3.00 |
| IPA | Qs |
| Pharmatose DCL 11 | 50.00 |
| Aer-O-Sil | 3.00 |
| Magnesium Stearate | 3.00 |
| TOTAL | 300.00 |

### EXAMPLE 3

Tablets having the composition shown in Table 5 were prepared in substantially the same manner as in Example 1.

TABLE 5

| | Qty/Tab |
|---|---|
| INGREDIENTS | (mg) |
| Alprazolam | 3.00 |
| Pharmatose 200M | 137.60 |
| Hydroxypropyl methylcellulose | 65.00 |
| (Methocel ® K4M CR) | |
| Viscosity by Ubbelhode | |
| 3000-5600 cps | |
| (nominal value 4000 cps) | |
| Hydroxypropyl methylcellulose | 35.00 |
| (Methocel ® E4M) | |
| Viscosity by Ubbelhode | |
| 3000-5600 cps | |
| (nominal value 4000 cps) | |
| D&C Yellow No. 10 | 0.20 |
| FD&C Blue No. 2 | 0.20 |
| Povidone K-25 | 3.00 |
| IPA | Qs |
| Pharmatose DCL 11 | 50.00 |
| Aer-O-Sil | 3.00 |
| Magnesium Stearate | 3.00 |
| TOTAL | 300.00 |

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A sustained release pharmaceutical composition in solid dosage form comprising (a) a therapeutically effective amount of one or more active pharmaceutical ingredients; (b) a first high viscosity release retarding cellulose ether; and (c) a second high viscosity release retarding cellulose ether, wherein the first and second high viscosity release retarding cellulose ethers are of the same material.

2. The composition of claim 1, wherein the active pharmaceutical ingredient is selected from the group consisting of alprazolam, acetazolamide and mixtures thereof.

3. The composition of claim 1, wherein the first and second high viscosity release retarding cellulose ethers have a viscosity of greater than about 400 centipoise (cP).

4. The composition of claim 1, wherein the first and second high viscosity release retarding cellulose ethers have a viscosity of at least about 1500 cP.

5. The composition of claim 1, wherein the first and second high viscosity release retarding cellulose ethers have a viscosity of at least about 4000 cP.

6. The composition of claim 1, wherein the first high viscosity release retarding cellulose ether has a viscosity greater than or equal to about 1000 cP and the second high viscosity release retarding cellulose ether has a viscosity greater than or equal to about 5000 cP.

7. The composition of claim 1, wherein the first high viscosity release retarding cellulose ether has a viscosity greater than or equal to about 3500 cP and the second high viscosity release retarding cellulose ether has a viscosity greater than or equal to about 10,000 cP.

8. The composition of claim 1, wherein the first high viscosity release retarding cellulose ether has a viscosity greater than or equal to about 4000 cP and the second high viscosity release retarding cellulose ether has a viscosity greater than or equal to about 15,000 cP.

9. The composition of claim 1, wherein the first and second high viscosity release retarding cellulose ethers are selected from the group consisting of a hydroxypropyl methylcellulose (HPMC) polymer, hydroxypropyl cellulose (HPC) polymer, hydroxyethyl cellulose (HEC) polymer and mixtures thereof.

10. The composition of claim 1, wherein the first and second high viscosity release retarding cellulose ethers are a HPMC polymer.

11. The composition of claim 10, wherein the first and second high viscosity HPMC are of a type selected from the group consisting of 2208, 2906 and 2910.

12. The composition of claim 10, wherein the first high viscosity HPMC is of type 2208 and the second high viscosity HPMC is of type 2910.

13. The composition of claim 10, wherein the first high viscosity HPMC is of type 2208 and the second high viscosity HPMC is of type 2910 and the viscosity of the first and second high viscosity HPMC is the same.

14. The composition of claim 10, wherein the first high viscosity HPMC is of type 2208 and the second high viscosity HPMC is of type 2910 and the viscosity of the first and second high viscosity HPMC is different.

15. The composition of claim 1, wherein the first high viscosity release retarding cellulose ether is HPMC of type 2208 and the second high viscosity release retarding cellulose ether is HPMC of type 2910 and the viscosity of the first and second high viscosity HPMC is the same.

16. The composition of claim 1, wherein the amount of the first high viscosity release retarding cellulose ether is about 15 to about 35 weight percent and the amount of the second high viscosity release retarding cellulose ether is about 5 to about 20 weight percent.

17. The composition of claim 1, further comprising one or more pharmaceutically acceptable excipients.

18. The composition of claim 1, in the form of a tablet.

19. The composition of claim 18, having a total tablet weight of about 200 mg to about 500 mg.

20. A method of treating a CNS condition or disorder in a subject, the method comprising orally administering to the subject a therapeutically effective amount of the composition of claim 1.
